(19) United States
(12) Patent Application Publication
(10) Pub. No.: US 2016/0143822 A1
(43) Pub. Date: May 26, 2016

(54) EYELASH ORNAMENT COSMETIC

(71) Applicant: MITSUBISHI PENCIL COMPANY, LIMITED, Tokyo (JP)

(72) Inventors: Takayuki HAYAKAWA, Fujioka-shi (JP); Satoshi SAKUMA, Fujioka-shi (JP)

(73) Assignee: MITSUBISHI PENCIL COMPANY, LIMITED, Tokyo (JP)

(21) Appl. No.: 14/928,007

(22) Filed: Oct. 30, 2015

(30) Foreign Application Priority Data
Nov. 21, 2014 (JP) 2014-236436

(51) Int. Cl. A61K 8/06; A61Q 1/00; A61K 8/81

(52) U.S. Cl. CPC A61K 8/06; A61K 8/8141; A61Q 1/00; A61K 2800/412; A61K 2800/654

(57) ABSTRACT

Provided is an eyelash ornament cosmetic comprising at least a polymer emulsion and an extender wherein an average particle diameter of the extender is 1 μm or less, wherein droplets applied on the tips of eyelashes are dried and turned into fine spherical resin balls, and the tips of the eyelashes are ornamented to make it possible to provide the eyes with brilliantness and loveliness.

This Nonprovisional application claims priority under 35 U.S.C. §119(a) on Patent Application No. 2014-236436 filed in Japan on 21 Nov. 2014, the entire contents of which are hereby incorporated by reference.

### BACKGROUND OF THE INVENTION

1. Field of the Invention

The present invention relates to a cosmetic for ornamenting the tips of eyelashes. More specifically, the present invention relates to an eyelash ornament cosmetic which is applied on the tips of eyelashes in the form of droplets and which is dried and turned into fine spherical resin balls to ornament the tips of eyelashes.

2. Description of the Conventional Art

Methods in which eyelashes are curled or extended by a mascara have so far been usually used as cosmetics for ornamenting eyelashes.

Known are, for example, a method for enhancing a durability of curling by using a water-proof coating, and a method in which eyelashes are made look longer by blending fibers.

However, the methods described above are methods for efficiently using or emphasizing the original form of eyelashes, and they are not methods for presenting brilliantness and loveliness.

Also, a method in which vividness is given to eyes by applying a mascara having an unusual color on eyelashes is used as well. Brilliantness can be exhibited by the above method, however, the suitable situation is limited, and it is difficult to use ordinary.

To refer to, for example, patent document 1, a cosmetic for eyelashes which is prepared by blending an alkyl acrylate polymer emulsion, a moisturizer and a polyacrylate and which makes eyelashes voluminous by curling is known as a conventional cosmetic for eyelashes. In the above cosmetic for eyelashes, “curling power”, “smoothness” and “quick drying” are emphasized for evaluation, and is not focused on enabling specific ornament primarily.

Further, to refer to, for example, patent document 2, known is a cosmetic for eyelashes which contains an acrylic base film-forming polymer having a glass transition temperature of 80° C. or higher and a different film-forming polymer emulsion and which does not form lumps. It is described therein that the above cosmetic for eyelashes contains preferably powders having an average particle diameter of 1 to 20 μm and that spherical inorganic powders such as spherical silica are particularly preferred. Also in patent document 2, it is not focused on to enable specific ornament.
Patent document 1: Japanese Patent Application Laid-Open No. 2014/159386, claims, examples and others
Patent document 2: Japanese Patent Application Laid-Open No. 2008/50315, claims, paragraph [0032], examples and others

### SUMMARY OF THE INVENTION

In light of the existing situations of the conventional art described above, an object of the present invention is to provide an eyelash ornament cosmetic which can easily provide eyes with brilliantness and loveliness without hesitation by applying specific makeup such as spherical decoration on eyelashes.

### [Means for Solving the Problems]

Intense investigations repeated by the present inventors have resulted in finding that an eyelash ornament cosmetic which maintains a three-dimensional form even after dried and which meets the object described above is obtained by comprising at least a polymer emulsion and an extender wherein an average particle diameter of the extender is a specific value or lower, and thus, the present inventors have come to complete the present invention.

That is, the present invention resides in the following items (1) to (6).
(1) An eyelash ornament cosmetic comprising at least a polymer emulsion and an extender wherein an average particle diameter of the extender is 1 μm or less.
(2) The eyelash ornament cosmetic as described in the above item (1), wherein the extender is an acrylic resin powder.
(3) The eyelash ornament cosmetic as described in the above item (1) or (2), wherein a total solid content of the polymer emulsion and the extender is 20 to 70% by mass based on a total amount of the cosmetic.
(4) The eyelash ornament cosmetic as described in any one of the above items (1) to (3), wherein the component of the polymer emulsion is an acrylic resin emulsion.
(5) The eyelash ornament cosmetic as described in any one of the above items (1) to (4), wherein the polymer emulsion has a glass transition temperature, Tg of 50° C. or lower.
(6) The eyelash ornament cosmetic as described in any one of the above items (1) to (5), wherein the eyelash ornament cosmetic has a viscosity of 1000 to 20000 mPa·s at a shear rate of 3.83 s⁻¹ at 25° C.

### [Effect of the Invention]

According to the present invention, provided is an eyelash ornament cosmetic which can readily be applied on the eyelashes by the user in the form of droplets and which can be attached on the eyelashes in the form of resin balls having various colors by drying the droplets while maintaining the three-dimensional form thereof to make it possible to provide the eyes with brilliantness and loveliness.

### DETAILED DESCRIPTION OF THE PREFERRED EMBODIMENTS

The embodiments of the present invention shall be explained below in detail.

The eyelash ornament cosmetic of the present invention is characterized by comprising at least a polymer emulsion and an extender wherein an average particle diameter of the extender is 1 μm or less.

The polymer emulsion used in the present invention is added for the purpose of forming and maintaining the spherical form of the cosmetic. The polymer emulsion prescribed in the present invention means an emulsion which is dispersed by such as a surfactant and has a film-forming property.

The polymer emulsion used in the present invention includes such as an acrylic resin emulsion, a urethane resin emulsion, and a vinyl acetate resin emulsion.

In the above polymer emulsion, a glass transition temperature Tg shall not specifically be restricted, however, the glass transition temperature Tg is preferably 50° C. or lower, more preferably 40° C. or lower from the viewpoints of a hardness after drying, maintaining the form and forming the spheres.

In the present invention including examples described later, the “glass transition temperature Tg” means temperature at which when an amorphous solid is heated, the hard solid having no fluidity like a crystal at low temperature is reduced rapidly in a rigidity and a viscosity and increased in a fluidity in some narrow temperature range, that is, temperature at which glass transition is caused.

An acrylic resin emulsion is desirably used as the preferred polymer emulsion from the viewpoints of a hardness and compatibility with the extender.

The acrylic resin emulsion which can be used includes, for example, alkyl acrylate copolymer emulsions, acrylic ester-methacrylic ester copolymer emulsions, alkyl acrylate.styrene copolymer emulsions, vinyl acetate.alkyl acrylate copolymer emulsions.

The polymer emulsion which can specifically be used includes, in the case of the acrylic resin emulsion, commercially available such as Yodosol GH34F, an alkyl acrylate copolymer emulsion having Tg of lower than 50° C. manufactured by Akzo Nobel N.V.; Vinysol 1076DM, an alkyl acrylate copolymer emulsion having Tg of 50° C. or higher manufactured by Daido Chemical Corporation; and Daitosol 5000 AD, an alkyl acrylate copolymer emulsion having Tg of lower than 50° C. manufactured by Daito Kasei Kogyo Co., Ltd.; in the case of the urethane resin emulsion, such as BAYCUSAN C1000 having Tg of lower than 50° C. manufactured by Bayer Material Science A. G., and BAYCUSAN C1004 having Tg of lower than 50° C. manufactured by Bayer Material Science A. G., and in the case of the vinyl acetate resin emulsion, such as Vinyblan

GV5651 having Tg of lower than 50° C. manufactured by Nissin Chemical Industry Co., Ltd.

A content of the above polymer emulsion is varied in the relation to the extender used, and the content is 1 to 60% by mass, preferably 5 to 40% by mass in terms of a solid concentration based on a total amount of the cosmetic. Hereinafter, “% by mass” is referred to merely as “%”.

The extender having an average particle diameter of 1 μm or less which is used in the present invention is added for the purpose of forming a spherical shape.

The extender which can be used includes, for example, at least one of resin particles such as acrylic resin powders, urethane resin powders and the like, talc, mica, barium sulfate, and silicon dioxide.

The above extenders have to have an average particle diameter of 1 μm or less, preferably 0.005 to 1.0 μm or 5 to 1000 nm from the viewpoints of maintaining the form and providing the viscosity.

The “average particle diameter” of the extender prescribed in the present invention means a number average particle diameter. The “average particle diameter” is obtained as the average of the measured diameters for the spherical and clear image of ten sample particles around the center in a captured magnified image. The magnified image is observed by a Scanning Electron Microscope for the sample particle spread on a tape.

The acrylic resin powders are desirably used as the preferred extenders from the viewpoint of a compatibility with the polymer emulsion.

Also, the resin particles such as acrylic resin powders, and urethane resin powders mean particles which do not have a film-forming property and are not dispersed.

The acrylic resin powder which can be used includes such as polymethyl methacrylate resin particles and alkyl acrylate.styrene copolymer particles, and the acrylic resin powder includes, to be specific, commercially available MP-2200, a polymethyl methacrylate resin particles, having an average particle diameter of 300 to 500 nm manufactured by Soken Chemical & Engineering Co., Ltd., and MX-80H3wT, an acrylic resin particles having an average particle diameter of 0.8 μm manufactured by Soken Chemical & Engineering Co., Ltd.

The above extenders can be used alone or in a mixture of two or more kinds thereof.

A content of the above extender is adjusted in relation to the foregoing polymer emulsion used. A total content of the polymer emulsion and the extender used is 15 to 75%, preferably 20 to 70% and particularly preferably 20 to 60% in terms of a solid concentration based on a total amount of the cosmetic from the viewpoints of the fluidity and maintaining the form.

If a total content of the polymer emulsion and the extender used is less than 15% in terms of a solid concentration, the solid matter is short, and the form is deflated and cannot be maintained. On the other hand, if the content exceeds 75%, the fluidity markedly gets worse, and the form cannot be maintained. Accordingly, both are not preferred.

The eyelash ornament cosmetic of the present invention contains the respective components described above and can contain, if necessary, a suitable amount of a colorant in order to provide a stereoscopic color pattern and a color shape.

Organic pigments, inorganic pigments, pearl pigments, and bright pigments which are usually used for cosmetics can be used as the usable colorant.

Capable of being listed are the organic pigments such as blue color No. 1 Al lake, red color No. 202, red color No. 226, No. 228, blue color No. 404, red color No. 220, yellow color No. 401, yellow color No. 205, blue color No. 201, blue color No. 204, yellow color No. 4 Al lake, yellow color No. 203 Al lake, and red color No. 104 Al lake, the inorganic pigments such as carbon black, Prussian blue, red iron oxide, yellow iron oxide, black iron oxide, and titanium oxide, and the pearl pigments such as mica, mica titanium, carmine-coated mica titanium, carmine.Prussian blue-coated mica titanium, black iron oxide-coated mica titanium, black iron oxide.carmine-coated mica titanium, black iron oxide.Prussian blue-coated mica titanium, blue-coated mica titanium, red iron oxide-coated mica, red iron oxide-coated mica titanium, red iron oxide.carmine-coated mica titanium, red iron oxide.black iron oxide-coated mica titanium, red iron oxide.Prussian blue-coated mica titanium, and red iron oxide.black iron oxide.Prussian blue-coated mica titanium.

A content of the above colorant is 0 to 10%, preferably 0 to 5% based on a total amount of the cosmetic in order to obtain the suitable stereoscopic color pattern, color shape and brilliantness.

Also, the eyelash ornament cosmetic of the present invention can contain water such as refined water, distilled water, ion-exchanged water and purified water as the balance. Further, it can contain a water-soluble organic solvent for the purpose of controlling the drying property, and other optional components, for example, other various kinds of the respective components such as a pH modifier, a neutralizer, a UV absorber, a scattering agent, a moisturizer, a perfume, an antioxidant, an antiseptic agent, a chelating agent, a defoamant, and various extracts.

The organic solvent which can be used includes, for example, alcohols such as ethanol, phenoxyethanol, and 2-propanol, glycols such as 1,3-butylene glycol and pentylene glycol, and glycerins such as glycerin and ethylhexyl glycerin.

A content of the above organic solvents is 0 to 20%, preferably 0.1 to 20% based on a total amount of the cosmetic in order to obtain the suitable drying property.

The eyelash ornament cosmetic of the present invention has a viscosity of preferably 1000 to 50000 mPa·s, more preferably 1000 to 20000 mPa·s and particularly preferably 1500 to 5000 mPa·s at a shear rate of 3.83 s⁻¹ at 25° C. from the viewpoint of forming the three-dimensional shape.

If the above viscosity is less than 1000 mPa·s, the droplets cannot be held on the eyelashes by completing drying, and on the other hand, if the viscosity exceeds 50000 mPa·s, the spherical droplets cannot be formed in certain cases.

The viscosity ranges described above can be modified by effectively combining the polymer emulsion, the extender, water and the like each described above in the ranges of the contents described above and by using a thickener if necessary.

The eyelash ornament cosmetic of the present invention can be prepared by an ordinary method, and the eyelash ornament is filled in an applicator for eyelashes and applied on the eyelashes.

Capable of being used are applicators such as, for example, brushes prepared by rounding a wire around fibers usually used to fix them, applicators of a comb shape and a coil shape, resin-molded brushes molded to a brush form by using a resin, brushes, flocks, and rod-like applicators.

The eyelash ornament cosmetic of the present invention thus constituted contains at least the polymer emulsion and the extender wherein an average particle diameter of the extender is 1 μm or less, so that the user can readily apply droplets on eyelashes. Further, the droplets can be dried to form the resin spheres of various colors on the eyelashes to result in obtaining an eyelash ornament cosmetic which can provide eyes with brilliantness and loveliness.

### EXAMPLES

Next, the present invention shall be explained in further details with reference to examples, however, the present invention shall not be restricted by the following examples and the like.

### Examples 1 to 7 and Comparative Examples 1 to 3

The components were mixed and dispersed in blend prescriptions with total amount of 100% shown in the following Table 1 by means of a planetary stirring device, a planetary mixer, a homomixer or a disperser to prepare the respective eyelash ornament cosmetics.

The respective eyelash ornament cosmetics obtained above in Examples 1 to 7 and Comparative Examples 1 to 3 were used to measure the respective viscosities at a shear rate of 3.83 s⁻¹ at 25° C. by the following measuring method. Further, the adhesiveness onto eyelashes, the three-dimensional shape-forming property and a transparency of the dried spheres were evaluated by the following evaluation methods. The results thereof are shown in the following Table 1.

### Measuring Method of Viscosity:

The respective eyelash ornament cosmetics obtained above were used to measure a viscosity at the prescribed shear rate at 25° C. by means of a cone plate viscometer of an EHD type viscometer among TV-30 viscometers with a standard cone plate manufactured by Tokimec Inc.

### Measuring Method of Adhesiveness Onto Eyelashes:

The respective eyelash ornament cosmetics were charged into an exclusive vessel, applied on eyelashes and dried to evaluate an adhesiveness onto eyelashes, a three-dimensional shape-forming property and a transparency of the dried spheres according to the following respective evaluation criteria. They were evaluated by one exclusive panel.

### Evaluation Criterion of Adhesiveness Onto Eyelashes:

A: Spheres applied on eyelashes were not dropped off whole one day
B: Spheres applied on eyelashes were not dropped off whole one day but readily dropped off by a finger touch
C: Spheres applied on eyelashes were dropped off in daily life
D: Spheres were not adhered on eyelashes

### Evaluation Criterion of Three-Dimensional Shape-Forming Property:

A: Fine spherical ornaments were always formed after drying
B: Three-dimensional ornaments having any form were always formed after drying
C: Three-dimensional ornaments having any form were partially formed after drying
D: Three-dimensional ornaments were not formed after drying

### Evaluation Criterion of Transparency of the Dried Spheres:

A: Ornaments after dried were transparent and looked brilliant by refraction of light
B: Ornaments after dried were opaque but glossy and looked bright
C: Ornaments after dried were opaque and looked cloudy
D: Three-dimensional ornaments were not formed after dried

TABLE 1
(total amount: 100% by mass)

| | | | Example | | | | |
|---|---|---|---|---|---|---|---|
| | Blend component | Material name | 1 | 2 | 3 | 4 | 5 |
| Polymer | Acrylic resin | Yodosol | 60 | 60 | | | 60 |
| emulsion | emulsion | GH34F *1 | | | | | |
| | Acrylic resin | Vinysol | | | 60 | | |
| | emulsion | 1076DM *2 | | | | | |
| | Urethane resin | BAYCUSAN | | | | 60 | |
| | emulsion | C1000 *3 | | | | | |
| Extender | Acrylic resin | MP2200 *4 | 30 | 29 | 30 | 30 | |
| | powder | | | | | | |
| | Acrylic resin | Techpolymer | | | | | |
| | powder | MB-4C *5 | | | | | |
| | Silica | RY200 *6 | | | | | 30 |
| Colorant | Carbon black | UNIPURE BLACK | 0.2 | | 0.2 | 0.2 | 0.2 |
| | | LC902 *7 | | | | | |
| | Pearl pigment | MP-1001 *8 | | 1 | | | |
| Solvent | 1,3-Butylene | | 5 | 5 | 5 | 5 | 5 |
| | glycol | | | | | | |
| | Pentylene glycol | | 2 | 2 | 2 | 2 | 2 |
| | Phenoxyethanol | | 0.5 | 0.5 | 0.5 | 0.5 | 0.5 |
| | Ethylhexyl | | 0.2 | 0.2 | 0.2 | 0.2 | 0.2 |
| | glycerin | | | | | | |
| | Water as balance | Purified water | 2.1 | 2.3 | 2.1 | 2.1 | 2.1 |
| Total solid content of polymer emulsion and | | | 55.2 | 54.2 | 50.76 | 54.0 | 55.2 |
| extender | | | | | | | |
| Viscosity (25° C., shear rate of 3.83 s⁻¹, mPa · s) | | | 2000 | 2200 | 2500 | 3000 | 30000 |
| Evaluation | Adhesiveness onto eyelashes | | A | A | C | A | B |
| | 3D shape-forming property | | A | A | B | A | C |
| | Transparency of dried spheres | | A | A | A | C | C |
| | | | | | Comparative | | |
| | | | Example | | Example | | |
| | Blend component | Material name | 6 | 7 | 1 | 2 | 3 |
| Polymer | Acrylic resin | Yodosol | 20 | 37 | 90 | | 60 |
| emulsion | emulsion | GH34F *1 | | | | | |
| | Acrylic resin | Vinysol | | | | | |
| | emulsion | 1076DM *2 | | | | | |
| | Urethane resin | BAYCUSAN | | | | | |
| | emulsion | C1000 *3 | | | | | |
| Extender | Acrylic resin | MP2200 *4 | 10 | 55 | | 50 | |
| | powder | | | | | | |
| | Acrylic resin | Techpolymer | | | | | 30 |
| | powder | MB-4C *5 | | | | | |
| | Silica | RY200 *6 | | | | | |
| Colorant | Carbon black | UNIPURE BLACK | | 0.2 | 0.2 | 0.2 | 0.2 |
| | | LC902 *7 | | | | | |
| | Pearl pigment | MP-1001 *8 | | | | | |
| Solvent | 1,3-Butylene | | 5 | 5 | 5 | 5 | 5 |
| | glycol | | | | | | |
| | Pentylene glycol | | 2 | 2 | 2 | 2 | 2 |
| | Phenoxyethanol | | 0.5 | 0.5 | 0.5 | 0.5 | 0.5 |
| | Ethylhexyl | | 0.2 | 0.2 | 0.2 | 0.2 | 0.2 |
| | glycerin | | | | | | |
| | Water as balance | Purified water | 62.3 | 0.1 | 2.1 | 42.1 | 2.1 |

| Total solid content of polymer emulsion and | | 18.4 | 70.54 | 37.8 | 50.0 | 55.2 |
|---|---|---|---|---|---|---|
| extender | | | | | | |
| Viscosity (25° C., shear rate of 3.83 s⁻¹, mPa · s) | | 300 | 50000 | 30 | 50000 | 600 |
| Evaluation | Adhesiveness onto eyelashes | C | B | C | D | D |
| | 3D shape-forming property | C | C | D | B | C |
| | Transparency of dried spheres | A | C | D | D | C |

*1: Solid concentration: 42%, Tg < 50° C., alkyl acrylate copolymer emulsion, manufactured by Akzo Nobel N.V.
*2: Solid concentration: 34.6%, Tg ≧ 50° C., alkyl acrylate copolymer emulsion, manufactured by Daido Chemical Corporation
*3: Solid concentration: 40%, Tg < 50° C., urethane resin emulsion, manufactured by Bayer Material Science A.G.
*4: Average particle diameter: 300 to 500 nm, polymethyl methacrylate particles, manufactured by Soken Chemical & Engineering Co., Ltd.
*5: Average particle diameter: 4 μm, polymethyl methacrylate particles, manufactured by Sekisui Plastics Co., Ltd.
*6: Average particle diameter: 7 nm, manufactured by Nippon Aerosil Co., Ltd.
*7: Manufactured by Sensient Technologies Corporation
*8: Manufactured by Merck KGaA

As apparent from the results summarized in Table 1 shown above, it has become clear that the eyelash ornament cosmetics falling in the scope of the present invention as Examples 1 to 7 are excellent in an adhesiveness onto eyelashes, a three-dimensional shape-forming property and a transparency of the dried spheres as compared with the eyelash ornament cosmetics out of the scope of the present invention as Comparative Examples 1 to 3.

To observe individually the comparative examples, the extender was not added in Comparative Example 1; the polymer emulsion was not added in Comparative Example 2; and an average particle diameter of the extender of exceeding 1 μm is out of the range for the present invention in Comparative Example 3. It has been found that the effects of the present invention cannot be exerted in all comparative examples.

1. An eyelash ornament cosmetic comprising at least a polymer emulsion and an extender wherein an average particle diameter of the extender is 1 μm or less.

2. The eyelash ornament cosmetic as described in claim 1, wherein the extender is an acrylic resin powder.

3. The eyelash ornament cosmetic as described in claim 1, wherein a total solid content of the polymer emulsion and the extender is 20 to 70% by mass based on a total amount of the cosmetic.

4. The eyelash ornament cosmetic as described in claim 1, wherein the polymer emulsion is an acrylic resin emulsion.

5. The eyelash ornament cosmetic as described in claim 1, wherein the polymer emulsion has a glass transition temperature Tg of 50° C. or lower.

6. The eyelash ornament cosmetic as described in claim 4, wherein the acrylic resin emulsion has a glass transition temperature Tg of 50° C. or lower.

7. The eyelash ornament cosmetic as described in claim 1, wherein the eyelash ornament cosmetic has a viscosity of 1000 to 20000 mPa·s at a shear rate of 3.83 s⁻¹ at 25° C.

8. The eyelash ornament cosmetic as described in claim 4, wherein a total solid content of the polymer emulsion and the extender is 20 to 70% by mass based on a total amount of the cosmetic.

9. The eyelash ornament cosmetic as described in claim 5, wherein a total solid content of the polymer emulsion and the extender is 20 to 70% by mass based on a total amount of the cosmetic.

10. The eyelash ornament cosmetic as described in claim 6, wherein a total solid content of the polymer emulsion and the extender is 20 to 70% by mass based on a total amount of the cosmetic.

11. The eyelash ornament cosmetic as described in claim 3, wherein the eyelash ornament cosmetic has a viscosity of 1000 to 20000 mPa·s at a shear rate of 3.83 s⁻¹ at 25° C.

12. The eyelash ornament cosmetic as described in claim 8, wherein the eyelash ornament cosmetic has a viscosity of 1000 to 20000 mPa·s at a shear rate of 3.83 s⁻¹ at 25° C.

13. The eyelash ornament cosmetic as described in claim 9, wherein the eyelash ornament cosmetic has a viscosity of 1000 to 20000 mPa·s at a shear rate of 3.83 s⁻¹ at 25° C.

14. The eyelash ornament cosmetic as described in claim 10, wherein the eyelash ornament cosmetic has a viscosity of 1000 to 20000 mPa·s at a shear rate of 3.83 s⁻¹ at 25° C.
